(19) United States
(12) Plant Patent Application Publication
(10) Pub. No.: US 2014/0345026 P1
(43) Pub. Date: Nov. 20, 2014

(54) Salvia plant named 'Eggben006'

(50) ;

(71) Applicant: Howard Bentley, Wonga Park (AU)

(72) Inventor: Howard Bentley, Wonga Park (AU)

(21) Appl. No.: 13/986,589

(22) Filed: May 15, 2013

(51) Int. Cl. A01H 5/00

(52) U.S. Cl. PLT/475

(57) ABSTRACT

A new and distinct Salvia cultivar named ‘EGGBEN006’ is disclosed, characterized by a dense, bushy plant habit and bright white flowers. The new variety is a Salvia, normally produced as an outdoor garden or container plant.

### LATIN NAME OF THE GENUS AND SPECIES

Salvia gregii×microphylla

### VARIETY DENOMINATION

‘EGGBEN006’

### BACKGROUND OF THE INVENTION

The new Salvia cultivar is a product of a planned breeding program conducted by the inventor, Steve Eggleton, in Wonga Park, Victoria, Australia. The objective of the breeding program was to produce new Salvia varieties with denser plant habits, strong garden performance, in a range of flower colors. The controlled pollination resulting in this new variety was made during March and April of 2008.

The parent variety is Salvia gregii×microphlla ‘EGGBEN003’, U.S. Plant patent application Ser. No. 13/506,161. The parent variety was self-pollinated to produce an F2 generation of seed. This seed was collected, sown and grown outdoors under field conditions. The new variety was identified as a potentially interesting selection in March of 2010, at a research greenhouse in Wonga Park, Australia. Further observations and evaluations were made throughout 2010 and 2011.

Asexual reproduction of the new cultivar ‘EGGBEN006’ by vegetative cuttings was first performed during 2010, at a research greenhouse in Wonga Park. Subsequent propagation has shown that the unique features of this cultivar are stable and reproduced true to type on successive generations.

### SUMMARY OF THE INVENTION

The cultivar ‘EGGBEN006’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘EGGBEN006’ These characteristics in combination distinguish ‘EGGBEN006’ as a new and distinct Salvia cultivar:
1. Bushy plant habit.
2. Bright white flower color
3. Floriferous plants
4. Dense plant habit.

### Parent Comparison

Plants of the new cultivar ‘EGGBEN006’ are similar to plants of the parent, in most horticultural characteristics, however, plants of the new cultivar ‘EGGBEN006’ bright white flowers compared to the creamy white flowers of the parent. Additionally, the new variety typically shows weak or no anthocyanin coloration in the stems, whereas the parent variety has strong anthocyanin coloration of the stems.

### Commercial Comparison

Plants of the new cultivar can be compared to the commercial variety Salvia jamensis ‘Moonlight Serenade’, unpatented. The two Salvia varieties are very similar in many characteristics, however, the new variety produces bright white flowers whereas the comparator has creamy white flowers. Plants of the new variety are significantly denser than plants of ‘Midnight Serenade’.

### BRIEF DESCRIPTION OF THE PHOTOGRAPHS

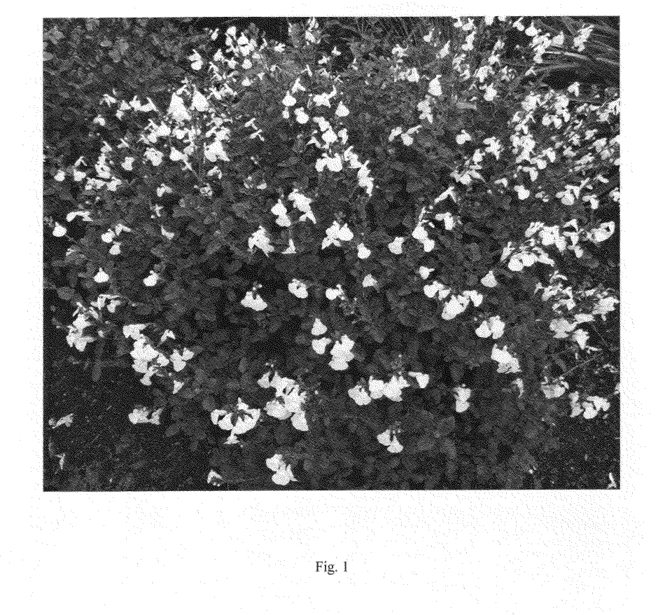

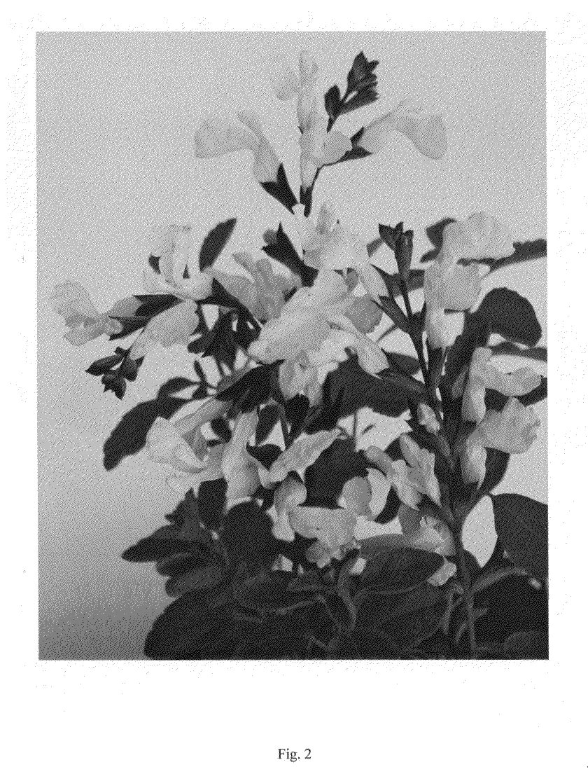

The accompanying photograph in FIG. 1 illustrates in full color a typical plant of ‘EGGBEN006’. FIG. 2 illustrates in full color typical flowers of ‘EGGBEN006’. The plants was grown outdoors, in Wonga Park, Australia. Age of the plant photographed is approximately 24 weeks from an unrooted cutting. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘EGGBEN006’ plants grown outdoors in Wonga Park, Australia. The growing temperature ranged from approximately 5° C. to 35° C. Measurements and numerical values represent averages of typical plant types.
Botanical classification: Salvia gregii×microphylla ‘EGGBEN006’
Propagation:
Time to initiate roots: About 5-8 days at approximately 25° C.
Root description: Fine, densely fibrous.
Time to produce a rooted cutting: About 21 days at 25° C.
Plant:
Growth habit: Upright, bushy annual.
Height: Approximately 28 cm to top of foliage. Approximately 35 cm to top of flowering plane. Measured from soil level of pot.
Plant spread: Approximately 25 cm.
Growth rate: Rapid.
Branching characteristics: Free-branching, Approximately 8 primary branches.
Length of primary lateral branches: Approximately 25 cm.
Diameter of lateral branches: Approximately 0.35 cm
Quantity of lateral branches: About 12 to 18.
Stem:
   Color.—Near RHS Green 137C.
   Pubescence.—Minute pubescence.
Internode length: Approximately 1.0 to 3.0 cm.
Age of plant described: Approximately 24 weeks from an unrooted cutting.
Foliage:
Leaf:
   Arrangement.—Opposite.
   Quantity.—Approximately 16 fully expanded per main branch.
   Average length.—Approximately 3.8 cm, including petiole.
   Average width.—Approximately 1.8 cm.
   Shape of blade.—Obovate.
   Apex.—Obtuse.
   Base.—Obtuse to nearly truncate.
   Attachment.—Stalked.
   Margin.—Very slightly crenate.
   Texture of top surface.—Matte, minute, coarse pubescence.
   Texture of bottom surface.—Matte, minute, coarse pubescence.
   Color.—Young foliage upper side: Near RHS Green 137A. Young foliage under side: Near RHS Green 137B. Mature foliage upper side: Near RHS Green 137A. Mature foliage under side: Near RHS Green 137B.
   Venation.—Type: Pinnate. Venation color upper side: Near RHS Yellow-Green 144A. Venation color under side: Near RHS Green 143C.
   Petiole.—Average Length: Approximately 1.1 cm. Diameter: Approximately 0.1 cm. Color: Near Green 143C.
Flower:
Natural flowering season: Flowering from early Spring through late Summer.
Inflorescence type and habit: Flowers arranged in verticillasters on spikes.
Flower longevity on plant: Individual flowers last approximately 1 week on the plant. Each spike lasts approximately 7 weeks with flowers.
Quantity of flowers: About 6 to 8 buds and 4 to 5 fully opened flowers per spike, at one time. Mature plants have approximately 10 to 20 spikes.
Spike size:
   Diameter.—Approximately 5.3 cm.
   Height.—Approximately 8.4 cm.
Individual flowers:
Size:
   Diameter.—Approximately 1.9 cm.
   Length.—Approximately 2.9 cm.
   Persistence.—Non-persistent.
   Fragrance.—Moderate, typical Salvia scent.
Corolla:
   Petal arrangement.—The corolla is sympetalous and typically bilabiate with 2 small, highly fused lobes forming an upper lip and 3 larger highly fused lobes forming a lower lip.
   Margin.—Nearly entire, slightly scalloped.
   Tip shape.—Upper lip tip retuse, lower lip tip rounded with scallops.
   Length.—Upper lip Approximately 1.1 cm, lower lip Approximately 1.5 cm. Tube length Approximately 1.8 cm.
   Width.—Upper lip Approximately 0.4 cm, lower lip Approximately 2.41 cm. Tube width Approximately 0.8 cm.
   Texture.—Upper lip: Highly pubescent on exterior surface. Interior surface glabrous Lower lip: Glabrous all surfaces.
Color: Upper Lip:
   When opening.—Inner surface: Near RHS White N155B, slight flush of Red 48C. Outer surface: Near RHS White N155B.
   Fully opened.—Inner surface: Near RHS White N155B, slight flush of Red 48C. Outer surface: Near RHS White N155B.
   Fading.—Inner surface: Near RHS White N155A Outer surface: Near RHS White N155A.
Color: Lower Lip:
   When opening.—Inner surface: Near RHS White N155A Outer surface: Near RHS White N155A.
   Fully opened.—Inner surface: Near RHS White N155A Outer surface: Near RHS White N155A.
   Fading.—Inner surface: Near RHS White N155A Outer surface: Near RHS White N155A.
Color: Tube:
   When opening.—Inner surface: Near RHS White N155B, slight flush of Red 48C. Outer surface: Near RHS White N155B, slight flush of Red 48C.
   Fully opened.—Inner surface: Near RHS White N155B Outer surface: Near RHS White N155B.
   Fading.—Inner surface: Near RHS White N155B Outer surface: Near RHS White N155B.
Bud:
   Shape.—Ovoid.
   Length.—Approximately 1.3 cm.
   Diameter.—Approximately 0.8 cm.
   Color.—Near RHS Yellow-White 158C, slight flush of Greyed-Red 180C.
Calyx:
   Length.—Approximately 1.3 cm.
   Diameter.—Approximately 0.8 cm.
   Shape.—Tubular.
Sepals:
   Shape.—Quantity per flower: 3, fused to form a tube. Unfused Apex: Length: Approximately 0.5 cm Width: Approximately 0.5 cm.
   Margin.—Entire.
   Apex.—Acute.
   Texture.—Pubescent.
   Color.—Immature: Near RHS Yellow-Green 144A, faint stripes near Grey-Purple 187A Mature: Near RHS Yellow-Green 144A, stripes near Grey-Purple 187A.
Peduncle:
   Length.—Average 3.4 cm.
   Diameter.—0.3 cm.
   Color.—Near RHS Green 137B.
   Orientation.—Upright, straight.
   Strength.—Strong.
   Texture.—Pubescent.
Pedicels:
   Length.—Average 0.2 cm.
   Diameter.—0.1 cm Color: Near RHS Green 137A.
   Orientation.—Straight, approximately 45 degree angle from attachment.
   Strength.—Flexible.
   Texture.—Pubescent.
Reproductive organs:
Stamens:
   Number.—2.
   Filament length.—Approximately 1.3, approximately 0.6 cm of length fused to petal.
   Filament color.—Near RHS White N155B.
Anthers:
   Shape.—Very narrowly elliptic.
   Length.—Approximately 3 mm.
   Color.—Near RHS Greyed-Yellow 161D.
   Pollen.—Moderate colored near Yellow-Orange 23B.
Pistil:
   Number.—1.
   Length.—Approximately 2.7 cm.
   Style.—Length: Approximately 2.7 cm Color: Near RHS White N155B.
   Stigma.—Shape: Linear, curled. Color: Near RHS Red-Purple 61A Ovary color: Near RHS Green-Yellow 1B.
Other characteristics:
Seeds and fruits: Not observed to date.
Disease/pest resistance: Neither resistance nor susceptibility to pathogens and pests common to Salvia have been observed.
Temperature tolerance: The new variety tolerates temperatures between 5 to 35° C.

What is claimed is:

1. A new and distinct cultivar of Salvia plant named ‘EGGBEN006’ as herein illustrated and described.
